(19) United States
(12) Patent Application Publication
(10) Pub. No.: US 2022/0313358 A1
(43) Pub. Date: Oct. 6, 2022

(54) ESTIMATING OPTICAL PROPERTIES OF SURGICAL TISSUE

(71) Applicant: Worcester Polytechnic Institute, Worcester, MA (US)

(72) Inventors: Andrea Arnold, Worcester, MA (US); Loris Fichera, Boston, MA (US)

(21) Appl. No.: 17/711,100

(22) Filed: Apr. 1, 2022

Related U.S. Application Data
(60) Provisional application No. 63/169,474, filed on Apr. 1, 2021.

(51) Int. Cl. A61B 18/20; A61N 5/067

(52) U.S. Cl. CPC A61B 18/20; A61N 5/067; A61B 2018/00636

(57) ABSTRACT

A method of assessing optical properties of surgical tissue by observing the tissue thermal response to laser irradiation includes exposing the tissue in a surgical region to a short laser pulse for triggering a localized temperature increase, and observing the triggered temperature increase by a thermal sensor. Optical properties of the tissue are then estimated, for determining laser surgery parameters for an eminent procedure, based on the observed temperature increase. In an example configuration, the thermal sensor is a thermal infrared camera, and estimation includes using an Ensemble Kalman Filter (EnKF) for comparing the temperature sensor data with the output of a computational laser-tissue interaction model.

### RELATED APPLICATIONS

This patent application claims the benefit under 35 U.S.C. § 119(e) of U.S. Provisional Patent App. No. 63/169,474, filed Apr. 1, 2021 entitled “ESTIMATING OPTICAL PROPERTIES OF SURGICAL TISSUE,” incorporated herein by reference in entirety.

### STATEMENT REGARDING FEDERALLY SPONSORED RESEARCH AND DEVELOPMENT

This invention was made, at least in part, with government support under Contract No. NSF/DMS-1819203, awarded by the National Science Foundation. The Government has certain rights in the invention.

### BACKGROUND

Lasers are an integral part of modern medicine, and their applications span across a wide variety of therapeutic areas. In minimally invasive surgery, lasers are frequently used to perform tissue cutting and ablation. Another important application is laser interstitial thermal therapy, where the goal is to thermally necrotize diseased tissue in-situ, e.g., to treat otherwise inoperable tumors. A key enabling factor of all these treatments is the ability to control how light is absorbed in the tissue and the temperature elevation that will be created as a result. An insufficient thermal dose will fail to achieve the desired effects and will therefore lead to an incomplete treatment; in contrast, an excessive thermal dose may cause charring (carbonization), a type of undesired thermal injury associated with scarring and a long recovery time. Existing laser-tissue interaction models require explicit knowledge of the optical characteristics of the tissue, including the absorption and scattering coefficients. Taken together, these two coefficients describe the optical penetration of light into the tissue and determine what fraction of light is absorbed.

### SUMMARY

A method of assessing optical properties of surgical tissue by observing the tissue thermal response to laser irradiation includes exposing the tissue in a surgical region to a short laser pulse for triggering a localized temperature increase, and observing the triggered temperature increase by a thermal sensor. Optical properties of the tissue are then estimated, for determining laser surgery parameters for an eminent procedure, based on the observed temperature increase. In an example configuration, the thermal sensor is a thermal infrared camera, and estimation includes using an Ensemble Kalman Filter (EnKF) for comparing the temperature sensor data with the output of a computational laser-tissue interaction model.

In recent years, several research groups have developed robotic systems to assist laser surgeons. The benefits of robotic assistance are manifold, including enhanced laser aiming accuracy, simpler laser control, and improved anatomical access. A natural extension of current research in robotic laser surgery is automation of laser actions. Laser cutting can be difficult to control for many physicians, as this process occurs in a contact-less fashion, making it impossible to use one's sense of touch to feel the cutting depth. Lasers can also cause secondary effects, most notably the creation of heat-affected zones, which are generally not desirable from a clinical standpoint, the occurrence of which can be difficult to anticipate or control. Motivated by these challenges, efforts have explored methods to automate laser ablation with the overarching objective of enhancing the accuracy and safety of laser surgeries.

A particular configuration implements laser ablation as a CNC-like cutting process, where repeated laser pulses are used to progressively remove material from the tissue surface until a prescribed ablation volume is created. For this approach to work, the laser ablation process first needs to be modeled; i.e., it is necessary to model the volume of tissue removed by each laser pulse, as well as the thermal effects created by the laser, so that the laser delivery can be properly planned and controlled. Unfortunately, building such models is not straightforward: the way in which laser light interacts with human tissue strongly depends on the tissue specific composition, including, among other things, the amount of water and blood content. These factors are well known to vary significantly among different types of tissue, and even among specimens of the same tissue type in different individuals

Lasers employ a focused beam of light having an intensity sufficient to irradiate a material for removal and excision by essentially burning irradiated material from the heat energy from photons in the beam. Configurations herein are based, in part, on the observation that lasers have an ability to manipulate, ablate ant cut surgical human tissue when movement and energy are precisely controlled. Unfortunately, conventional approaches to robotic guided laser surgery suffer from the shortcoming of thermal regulation for ensuring effective ablation (cutting and removal) while avoiding excessive heat in surrounding tissue, which can lead to undesirable charring. Accordingly, configurations herein detect and rapidly characterize the optical properties of biological tissue during robot-assisted laser surgery. The approach targets the identification of two specific tissue properties, namely, the absorption and scattering coefficients, which together indicate the optical penetration of light into the tissue for assessing the thermal impact on the tissue.

Identification of these coefficients is invoked to predict what fraction of laser light is absorbed under the form of heat, and what physical alterations will be created in the tissue as a result. Assessing the heat and effect on tissue contributes to the existing advances in robotic laser surgery by allowing robots to intelligently identify the type of tissue being operating on, where this information is significant for the automated planning and execution of laser actions.

In further detail, configurations herein provide a method for predicting tissue properties indicative of a thermal response of irradiated tissue, by directing an irradiation signal at tissue for a therapeutic effect. An optical sensor receives thermal images indicative of a temperature response of the irradiated tissue based on these optical properties of the irradiated tissue. The optical properties may be sensed by an infrared camera, and used to determine the temperature response and apply this temperature response to a thermal laser-tissue interaction model. This model is used for iteratively computing irradiation response coefficients indicative of an ablative effect on the irradiated tissue from the irradiation. The resulting computed response is then invoked for controlling the irradiation signal based on the irradiation response coefficients, such as the speed, direction and intensity of a robotically disposed laser.

### BRIEF DESCRIPTION OF THE DRAWINGS

The foregoing and other objects, features and advantages of the invention will be apparent from the following description of particular embodiments of the invention, as illustrated in the accompanying drawings in which like reference characters refer to the same parts throughout the different views. The drawings are not necessarily to scale, emphasis instead being placed upon illustrating the principles of the invention.

FIG. 1 is a context view of an irradiation or laser based system suitable for use with configurations herein;

FIG. 2 is a block diagram of the computation device or process of FIG. 1;

FIG. 3 shows an irradiation signal as in FIG. 1 intersecting a tissue region or sample;

FIGS. 4A-4E show graphs of constant parameter estimation using the filter of FIG. 2; and

FIGS. 5A-5E show graphs of time varying parameter estimation using the filter of FIG. 2.

### DETAILED DESCRIPTION

Configurations below describe implementation of an irradiation method, system and apparatus for laser based treatment of tissue. Alternate irradiation sources and radiation targets other than tissue may be utilized with the disclosed approach. The configurations below depict a particular approach and example using surgical approaches with human tissue, and exhibit differences in laser irradiation cutting, ablation and/or removal techniques with varied tissue types. The disclosed approach is easily deployable in a robotic mechanism for controlling and guiding a robotic actuator for surgical intervention responsive to the computed irradiation parameters as described below.

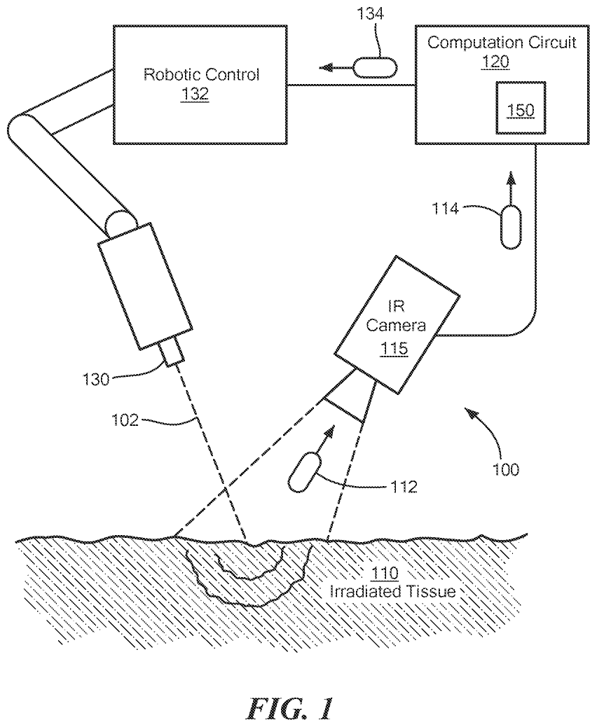

FIG. 1 is a context view of an irradiation or laser based system suitable for use with configurations herein. Referring to FIG. 1, in a medical irradiation environment 100, a method for predicting tissue properties indicative of a thermal response of irradiated tissue includes directing an irradiation signal 102 at tissue 110 for a therapeutic effect. An optical sensor 115 focused on the tissue 110 receives thermal images 112 indicative of a temperature response 114 of the irradiated tissue based on optical properties of the irradiated tissue 110. A computation circuit 120 applies the received temperature response 114 to a thermal laser-tissue interaction model 150 for computing irradiation response coefficients indicative of an ablative effect on the irradiated tissue from the irradiation. The irradiation signal 102 from a laser light source 130 has a degrading effect on the tissue 110. Intensity and movement of the light source 130, such as by a laser control 132, causes ablative effects including cutting, removal and eradication of the subject tissue. Precision direction from the laser control 132 is therefore significant in ensuring the resulting therapy is administered correctly, as excessive irradiation and cause tissue charring and insufficient energy will fall short of the desired goal. Accordingly, the laser control 132 directs the irradiation signal based on the computed irradiation response coefficients.

Conventional robotic systems for laser surgery cannot monitor and control the complex interactions that occur between the laser and the tissue. Configurations depicted below propose a method to identify the physical tissue properties that govern the dynamics of laser-tissue interactions. The disclosed approach focuses on the estimation of two quantities, namely, the absorption and scattering coefficients, whose knowledge is necessary to model the optical-thermal response of laser-irradiated tissue. Giving robots the ability to identify these coefficients is key to enable the automatic control of laser actions. The claimed approach estimates absorption and scattering using a system identification approach, i.e., by measuring the tissue temperature elevation created by a prescribed set of laser inputs, such as laser aiming, spot size, power and exposure time. Other laser inputs for control and intensity of the laser may also be programmed and directed; this set is exemplary.

Conventional uses of the absorption and scattering coefficients of tissue rely on the use of specialized and/or bulky optical equipment (e.g., integrating spheres) that is cumbersome to use in a surgical setting. Configurations herein, in contrast, characterize absorption and scattering based on the observation of the tissue thermal response to laser exposure. The disclosed approach operates as follows: given an initial guess of the unknown coefficients, first attempt to predict the tissue thermal response using a thermal laser-tissue interaction model; then update the tissue optical properties to minimize the difference between the predicted and observed tissue temperature. To perform the update, an Ensemble Kalman Filter (EnKF) may be employed. The EnKF is a type of Bayesian filtering algorithm for data assimilation.

The sequential nature of EnKFs enables the implementation of an online, or continual feedback estimation process, i.e., the absorption and scattering coefficients are progressively refined as more and more temperature observations become available over time. It should be noted that the implementation of the approach in the workflow of a robotic laser procedure only imposes the introduction of a thermal sensor to monitor the tissue, such as a miniaturized infrared thermocamera. No additional input or invasive diagnostics are imposed on the laser surgery patient.

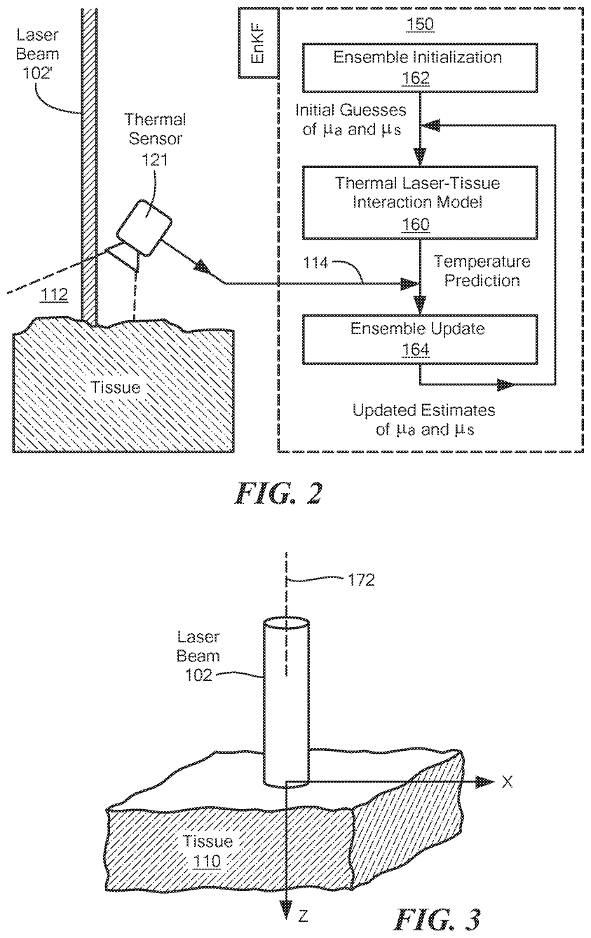

FIG. 2 is a block diagram of the computation device or process of FIG. 1. Referring to FIGS. 1 and 2, the laser control 132 directing an irradiation signal 102 at tissue 110 for a therapeutic effect from the laser light source 130. A thermal infrared camera 121 or other optical sensor 115 receives thermal images 112 indicative of a temperature response of the irradiated tissue based on optical properties of the irradiated tissue. An initial value or guess 162 is determined based on the tissue type, such as from Table I discussed further below. Other mechanisms for determining an initial value for one or more of the optical properties of the tissue may also be invoked. The computation circuit 120 applies the temperature response 114 to a thermal laser-tissue interaction model 160 for computing irradiation response coefficients indicative of an ablative effect on the irradiated tissue from the irradiation 102. The laser control 132 controls the irradiation signal 102 based on the irradiation response coefficients and other laser parameters 134 sent from the computation circuit 120.

The computation circuit 120 therefore receives thermal images 112 from the infrared camera 121 focused on an irradiated region of the tissue defined by the laser. In a particular configuration, the irradiation signal 102 is a laser and directing the irradiation signal includes actuating the laser for aiming and traversing the laser across the tissue 110.

The tissue properties include an absorption coefficient indicative of a fraction of photonic energy transferred to the tissue in the form of heat, and a scattering coefficient indicative of a direction change of a photon upon transferring the fraction of photonic energy. The resulting ablative effect causes heating for inducing removal or cutting of the tissue, including cutting, ablating, abrading and/or outright removal from the heat energy of the laser. As underscored above, careful application of the laser is needed to channel the potentially damaging effect in the proper manner to avoid damaging healthy or unexcised tissue in the surgical region.

The proposed approach identifies the tissue absorption and scattering coefficients based on Ensemble Kalman Filtering. In the EnKF framework, the unknown parameters of a system are modeled as stochastic variables whose probability density functions are encoded by a set of random realizations called an ensemble. Each time new sensor data becomes available, the ensemble is manipulated through a set of update rules to reflect the new probability distributions of the unknown parameters conditioned on the observed data. A more detailed formulation of the filter and a description of the ensemble update rules are described further below. Configurations herein use a version of EnKF capable of tracking time-varying parameters, which allows monitoring of the shift in the tissue optical properties that may occur during a laser procedure. Since these heating effects occur quickly in response to an ongoing laser surgical procedure, prompt response is important to identify appropriate laser control parameters in a timely manner.

Continuing to refer to FIG. 2, the tissue 110 is irradiated with a laser pulse or light beam 102, triggering a localized temperature increase which is observed by a thermal sensor 115 such as camera 121. For each observation, the EnKF compares the sensor data (temperature response 114 with the output of a laser-tissue interaction model 160, and iteratively updates the ensemble 164 in such a way to minimize the error between the model output and the measured tissue temperature. At any given time, the ensemble mean is used as an estimate of the unknown absorption and scattering coefficients, while the standard deviation provides a measure of uncertainty.

The thermal laser-tissue interaction model 160 defines a filter for computing optical penetration of the irradiation signal 102 into the tissue 110 and also the fraction of energy of the irradiation signal absorbed and manifested as heat by the irradiated tissue receiving the irradiation signal. It is important to define the role that the absorption and scattering coefficients play in the thermal response of laser-irradiated tissue. FIG. 2 shows the proposed approach for the identification of the tissue absorption and scattering coefficients, Ua and Us, respectively, during robot-assisted laser surgery. The laser light delivered to the tissue is absorbed under the form of heat, and the corresponding temperature increase is observed with a thermal sensor, i.e., an infrared thermal camera. The Ensemble Kalman Filter (EnKF) 150 is used to estimate the unknown coefficients based on the observed temperature dynamics.

At appropriate intervals, the computation circuit 120 iteratively revises the computed irradiation response coefficients based on the received thermal images. This includes comparing the temperature response based on the received thermal images with the computed irradiation response coefficients from a previous iteration. The filter performs tracking of time varying parameters based on a progression of the irradiation response coefficients in response to changes in the optical properties of the irradiated tissue 110 resulting from the irradiation signal 102.

FIG. 3 shows an irradiation signal as in FIG. 1 intersecting a tissue region or sample. Referring to FIGS. 1-3, tissue geometry as applied herein considers a rectangular block of tissue 110 exposed in air to an irradiation signal 102 such as a laser beam 102′. For simplicity, assume the tissue surface to be flat, and the laser beam to be perpendicular to the tissue. A Cartesian reference frame is established on the surface of the tissue, with the Z axis 170 coinciding with the optical axis 172 of the laser beam. Define the tissue temperature as a function T (x, y, z, t), where x, y, z are spatial coordinates, and t represents time. The tissue temperature can be calculated by solving the following differential equation:

$${c}_{v} \frac{\partial T}{\partial t} = k ( \frac{{\partial }^{2}}{\partial {x}^{2}} + \frac{{\partial }^{2}}{\partial {y}^{2}} + \frac{{\partial }^{2}}{\partial {z}^{2}} ) T + S , \qquad ( 1 )$$

where cᵥ is the volumetric heat capacity of the tissue (J cm⁻³ K⁻¹), k is the tissue thermal conductivity (W cm⁻¹ K⁻¹), and S is the volumetric power density (W cm⁻³). This latter term models the heat created by the laser in the tissue, and it is given by the product between the beam power (W) and the light absorption map A(x, y, z) (cm⁻³), which represents the fraction of light captured at any given location within the tissue volume.

To calculate the absorption map A, it is necessary to model the diffusion of light into the tissue. It is in these calculations that the coefficients of absorption μₐ and scattering μₛ (both having units of cm⁻¹) appear. Obtaining a closed form solution for the absorption map can be challenging, and this quantity is frequently calculated with a Monte Carlo method instead; the idea is to simulate the optical path of a large number of photons as a discrete random walk, and to keep track of where the photons deposit energy. The length of each step of the walk is sampled from a logarithmic distribution, i.e.,

$$s = \frac{- ln ( ζ )}{{μ}_{a} + {μ}_{s}} \qquad ( 2 )$$

where ζ is a computer-generated number sampled uniformly at random between 0 and 1. When a photon moves from one step to the next, its direction of travel will change due to scattering. This change in direction is modeled by means of an azimuthal component, sampled uniformly at random between 0 and 2π, combined with a deflection angle θ, which is typically modeled using the Henyey-Greenstein function, i.e.,

$$p ( cos ( θ ) ) = \frac{1 - {g}^{2}}{2 {( 1 + {g}^{2} - 2 g cos ( θ ) )}^{3 / 2}} , \qquad ( 3 )$$

with g being the expected value of cos(θ). This parameter is also known as the anisotropy factor, and for most biological tissues, its value has experimentally been determined to range between 0.7 and 0.99.

At each step of the walk, a photon loses a fraction of its energy due to absorption. A photon is terminated either when it escapes the tissue volume or when its residual energy level falls below some arbitrary small positive value €.

To estimate the absorption and scattering coefficients, μₐ and μₛ in (2), the computation circuit 120 uses an augmented EnKF 150. Given the observed sensor data, the goal is to formulate an approximation of the joint probability density function π(T, μₐ, μₛ) using a discrete sample. For conciseness of notation, we introduce a vector θ=(μₐ, μₛ), so that the probability density function can simply be written as π(T, θ).

Assume that we have a set of measurements dⱼ of the obtained sequentially by the thermal sensor at discrete times tⱼ, with j=1, . . . , M. Further assume that the data are corrupted by measurement errors. Let Tⱼ denote the temperature predicted by the laser-tissue interaction model at time j, and let θⱼ=(μ_{a,j}, μ_{s,j}) be a vector containing the parameter estimates at time j. The filtering process begins by drawing a random sample of size N from the prior distribution π(T₀, θ₀), which encodes any prior knowledge on the unknown coefficients. This forms the initial ensemble at time j=0. The filter then proceeds in a two-step updating scheme from time j to j+1, as described below.

### 1) Prediction Step: Given the Current Ensemble

Sⱼ={(Tⱼⁿ,θⱼⁿ)}ₙ₌₁^{N}

at time j, the prediction step of the EnKF updates the temperature values using a model approximation; i.e.,

T_{j+1|j}ⁿ=F(Tⱼⁿ,θⱼⁿ)+vⱼ₊₁ⁿ,n=1, . . . ,N (4)

Where F(Tⱼⁿ, θⱼⁿ) represents the numerical solution to (1) at time j+1, stored as a column vector, and the innovation

vⱼ₊₁ⁿ˜ [symbol] (0,C)

accounts for uncertainty in the forward prediction. The parameter values

θⱼⁿ.

are propagated forward using a random walk model of the form

θ_{j+1|j}ⁿ=θⱼⁿ+ξⱼ₊₁ⁿ,n=1, . . . ,N (5)

where

ξⱼ₊₁ⁿ˜ [symbol] (0,E)

with a prescribed covariance matrix E. Note that the parameter forward prediction in (5) is not necessary if the parameters are assumed to be constants; however, it is vital in tracking time-varying parameters. The predicted temperature and parameter values are augmented into vectors of the form

$${z}_{j + 1 \vert j}^{n} = [ \begin{matrix}{T}_{j + 1 \vert j}^{n} \\ {θ}_{j + 1 \vert j}^{n}\end{matrix} ] , n = 1 , … , N \qquad ( 6 )$$

which are used to compute the mean

$${\overline{z}}_{j + 1 \vert j} = \frac{1}{N} {\sum }_{n = 1}^{N} {z}_{j + 1 \vert j}^{n} \qquad ( 7 )$$

and the covariance

$${Γ}_{j + 1 \vert j} = \frac{1}{N - 1} {\sum }_{n = 1}^{N} ( {z}_{j + 1 \vert j}^{n} - {\overline{z}}_{j + 1 \vert j} ) {( {z}_{j + 1 \vert j}^{n} - {\overline{z}}_{j + 1 \vert j} )}^{T} \qquad ( 8 )$$

of the prediction ensemble.
2) Analysis Step: During the analysis step, the observed data dⱼ₊₁ are assimilated in producing the posterior ensemble, which is computed by

zⱼ₊₁ⁿ=_{j+1|j}ⁿ+Kⱼ₊₁(dⱼ₊₁ⁿ−G(z_{j+1|j}ⁿ)) (9)

for each n=1, . . . , N. Here

dⱼ₊₁ⁿ=dⱼ₊₁+wⱼ₊₁ⁿ,n=1, . . . ,N (10)

generates an ensemble of fictitious measurements around the observed data dⱼ₊₁, with

wⱼ₊₁ⁿ˜ [symbol] (0,D)

representing observation error; G is the observation function, which maps the predicted states and parameters to corresponding model observations; and Kⱼ₊₁ is the Kalman gain matrix, which contains cross-correlation information between the predicted model states and parameters. In this work, the observation function G in (10) is a linear mapping

G(z_{j+1|j}ⁿ)=P z_{j+1|j}ⁿ,n=1, . . . ,N (11)

where the projection matrix P picks out the tissue location at which the temperature is being measured, and the Kalman gain is computed by

Kⱼ₊₁=Γ_{j+1|j}P^{T}(PΓ_{j+1|j}P^{T}+D)⁻¹. (12)

Posterior ensemble statistics are computed similarly as in (7)-(8) using

zⱼ₊₁ⁿ.

The process repeats for j<M. Note that here we assume that temperature data are available at each time j; if measurements are not available at a subset of the filter time steps, the observation update can be neglected such that the prediction ensemble serves as the posterior at these steps.

To study the viability of the proposed method in estimating the unknown tissue absorption and scattering coefficients, we conducted a set of numerical experiments. Simulations were performed using the MATLAB® programming language (The MathWorks, Inc., Natick, Mass., well known in the art). Other computation packages may also be employed. The computation circuit 102 is a computer having a processor and memory suitable for executing and performing the simulations and related calculations, and for receiving the temperature response and rendering the results.

In each experiment, we simulate the setup previously illustrated in FIG. 1, where a laser pulse is applied to the surface of a sample of biological tissue. The laser beam has a uniform intensity profile, with a radius of 1 mm and a power of 0.5 W. The tissue is assumed to have the physical properties summarized in Table I. The dimensions of the tissue sample are 0.5 cm 0.5 cm 0.25 cm. For computational purposes, the tissue 110 block is discretized into a grid of 100×100×50 cubic elements, or voxels. The interaction between the laser beam and the tissue is monitored with a virtual thermal sensor, which provides superficial temperature measurements at a rate of 10 Hz. To simulate the presence of sensor noise, temperature measurements are altered with the addition of an error term e˜N (0, 0.01).

As noted, the goal is to estimate the absorption and scattering coefficients of the tissue given the thermal sensor data. At the beginning of each experiment, we initialize the EnKF with an ensemble of size N=50. The initial tissue temperature is set to 0° C., and the initial values for the unknown tissue coefficients are drawn from uniformly distributed priors, i.e., U (0.5θ\hat{ }, 2θ\hat{ }), where θ\hat{ } are the true coefficient values listed in Table I. To perform the temperature prediction step (§ II-B), the filter internally runs its own implementation of the laser-tissue interaction model described in section II-A with a coarser tissue grid (i.e. 20×20×10 voxels) than the one used to simulate the sensor data. The use of a coarser forward model helps to limit the computational complexity of the filter. While the absorption and scattering coefficients are unknown, we assume that the EnKF has knowledge of the other two tissue parameters in the thermal model, namely, the volumetric heat capacity, cᵥ, and thermal conductivity, k. We note that these additional parameters may not always be known a priori in a realistic setting, but reasonable approximations can generally be obtained using empirical models available in the laser tissue interactions literature.

TABLE I
TISSUE PHYSICAL PROPERTIES USED IN SIMULATION

| Symbol | Physical Variable | Units | Value Used |
|---|---|---|---|
| μa | Absorption Coefficient | cm−¹ | 1 |
| μs | Scattering Coefficient | cm−¹ | 100 |
| cv | Volumetric Heat Capacity | J/(cm−³ ° C.) | 3.76 |
| k | Thermal Conductivity | W/(cm ° C.) | 0.0037 |

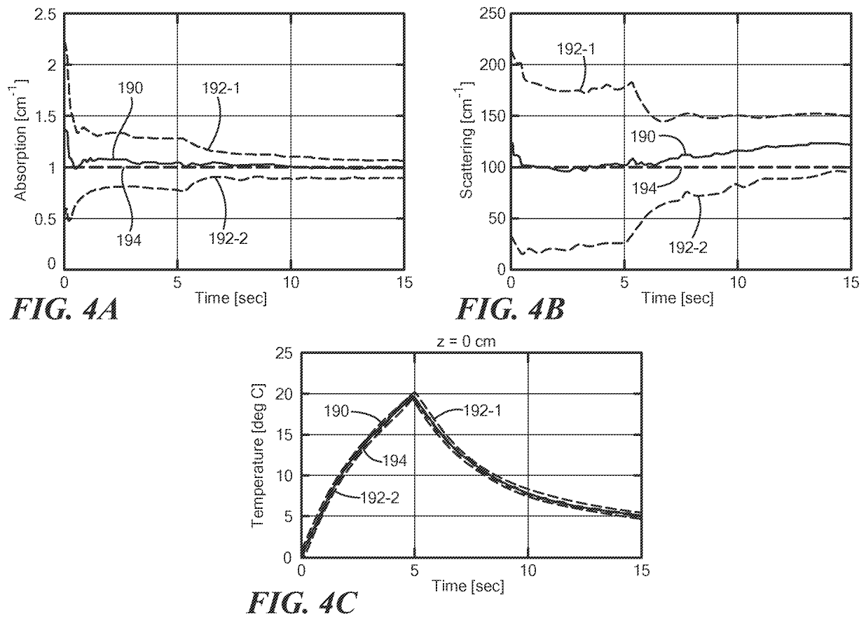

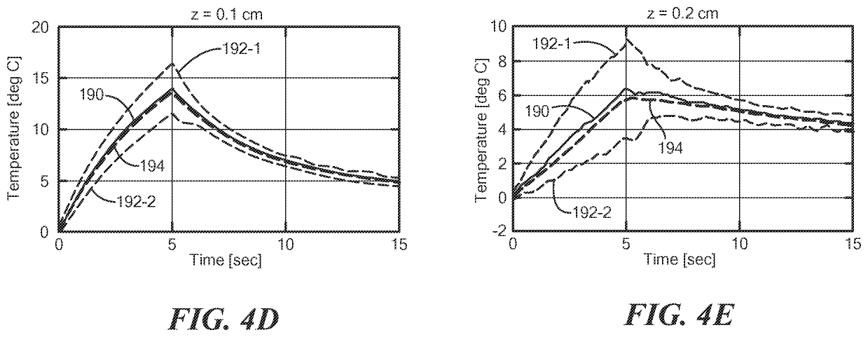

FIGS. 4A-E and 5A-E respectively describe two sets of numerical experiments: one in which the absorption and scattering coefficients remain constant over time; another in which the absorption and scattering coefficients shift during laser exposure (as may occur during a laser procedure). Each graph depicts a solid line 190 representing the EnKF mean, upper and lower dashed lines 192-1 . . . 192-2 showing an uncertainty bound flanking a true value shown by a dashed line 194.

FIGS. 4A-4E show graphs of constant parameter estimation using the filter of FIG. 2, depicted in a scenario in which the tissue optical properties remain constant during laser irradiation. FIGS. 4A-4W show application of a laser pulse for 5 seconds, then continue to observe the tissue temperature for 10 more seconds. We utilize the EnKF with the aim of estimating the true tissue absorption and scattering coefficients listed in Table I.

FIGS. 4A-4B show the resulting estimates of μₐ and μₛ, respectively, along with the corresponding tissue temperature estimates at three locations on and below the surface of the tissue; namely, we estimate the temperature profiles at the sensor location (x, y, z)=(0 cm, 0 cm, 0 cm) and at depths of 0.1 cm and 0.2 cm directly below (FIGS. 4A-4C, respectively). Note that the EnKF estimate in each plot is the ensemble mean, with uncertainty bounds given by ±2 standard deviations around the mean.

As seen in FIGS. 4A-4E, the EnKF provides an accurate estimate of the absorption coefficient, with uncertainty bounds shrinking over time. The estimate of the scattering coefficient drifts to a slightly higher value after the laser is turned off, with wider uncertainty bounds that do however contain the true parameter value. Further, while only observing data at the surface sensor location, the filter is able to well estimate the tissue temperature at the two locations tracked below the tissue surface, with wider uncertainty bounds as the location becomes farther from the surface.

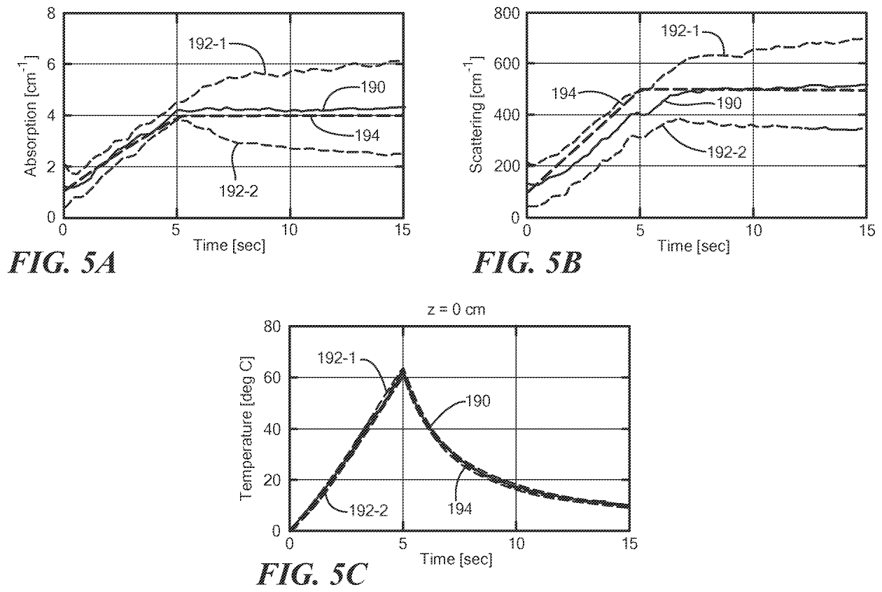

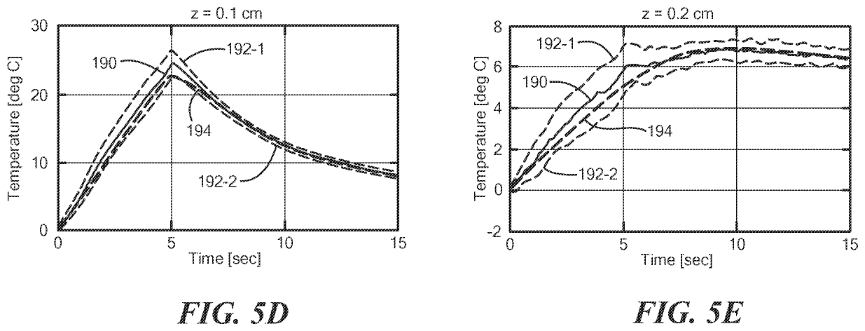

In this experiment depicted in FIGS. 5A-5E, the procedure described above is repeated, but simulating a scenario where the tissue optical properties change during laser exposure. Laser induced alterations in the absorption and scattering coefficients have been documented in prior literature. To simulate these shifts, we model both the absorption and scattering coefficients as continuous piecewise functions that increase linearly during the laser pulse and remain constant when the laser is turned off. More specifically, we let

$${μ}_{a} ( t ) = \{ \begin{matrix}0.6 t + 1 & if 0 \leq t \leq 5 \\ 4 & if 5 < t \leq 15\end{matrix} \\ and \qquad ( 13 )$$

$${μ}_{s} ( t ) = \{ \begin{matrix}80 t + 100 & if 0 \leq t \leq 5 \\ 500 & if 5 < t \leq 15\end{matrix} \qquad ( 14 )$$

respectively. It should be noted that the EnKF does not assume any knowledge of the relations above, and that the goal of this experiment is precisely to verify if the filter is able to track the absorption and scattering coefficients as their values change over time.

FIG. 5A-5E displays the resulting estimates of μₐ(t) and μₛ(t) (5D and 5E), along with the corresponding tissue temperature estimates at the three aforementioned locations (5A-5C, respectively). These results show that the filter is able to well track the change in absorption throughout the duration of the experiment. The increase in the scattering coefficient is more difficult to track during the laser pulse, but the filter is able to capture its behavior shortly after the laser is turned off. In both cases, the uncertainty bounds for the time-varying coefficients become increasingly wider once the laser is off. The estimates of the tissue temperature at and below the surface remain accurate throughout the experiment.

Those skilled in the art should readily appreciate that the programs and methods defined herein are deliverable to a user processing and rendering device in many forms, including but not limited to a) information permanently stored on non-writeable storage media such as ROM devices, b) information alterably stored on writeable non-transitory storage media such as floppy disks, magnetic tapes, CDs, RAM devices, and other magnetic and optical media, or c) information conveyed to a computer through communication media, as in an electronic network such as the Internet or telephone modem lines. The operations and methods may be implemented in a software executable object or as a set of encoded instructions for execution by a processor responsive to the instructions. Alternatively, the operations and methods disclosed herein may be embodied in whole or in part using hardware components, such as Application Specific Integrated Circuits (ASICs), Field Programmable Gate Arrays (FPGAs), state machines, controllers or other hardware components or devices, or a combination of hardware, software, and firmware components.

While the system and methods defined herein have been particularly shown and described with references to embodiments thereof, it will be understood by those skilled in the art that various changes in form and details may be made therein without departing from the scope of the invention encompassed by the appended claims.

What is claimed is:

1. A method for predicting tissue properties indicative of a thermal response of irradiated tissue, comprising:
directing an irradiation signal at tissue for a therapeutic effect;
receiving, from an optical sensor, thermal images indicative of a temperature response of the irradiated tissue based on optical properties of the irradiated tissue;
applying the temperature response to a thermal laser-tissue interaction model for computing irradiation response coefficients indicative of an ablative effect on the irradiated tissue from the irradiation; and
controlling the irradiation signal based on the irradiation response coefficients.

2. The method of claim 1 wherein the tissue properties include:
an absorption coefficient indicative of a fraction of photonic energy transferred to the tissue in the form of heat; and
and a scattering coefficient indicative of a direction change of a photon upon transferring the fraction of photonic energy.

3. The method of claim 1 wherein the ablative effect causes heating for inducing removal or cutting of the tissue.

4. The method of claim 1 wherein the irradiation signal is a laser and directing the irradiation signal includes actuating the laser for aiming and traversing the laser across the tissue.

5. The method of claim 4 further comprising receiving thermal images from an infrared camera focused on an irradiated region of the tissue defined by the laser.

6. The method of claim 1 wherein the thermal laser-tissue interaction model is a filter for computing optical penetration of the irradiation signal into the tissue and a fraction of energy of the irradiation signal absorbed by the irradiated tissue and manifested as heat by the irradiated tissue receiving the irradiation signal.

7. The method of claim 6 further comprising:
determining an initial value for one or more of the optical properties of the tissue;
computing the irradiation response coefficients based on the one or more initial values; and
iteratively revising the computed irradiation response coefficients based on the received thermal images.

8. The method of claim 7 wherein iteratively revising the irradiation response coefficients includes comparing the temperature response based on the received thermal images with the computed irradiation response coefficients from a previous iteration.

9. The method of claim 1 wherein the filter performs tracking of time varying parameters based on a progression of the irradiation response coefficients in response to changes in the optical properties of the irradiated tissue resulting from a pulsing of the irradiation signal.

10. A computer guided laser device for performing surgical manipulations based on method for predicting tissue properties indicative of a thermal response of irradiated tissue, comprising:
a laser operative for directing an irradiation signal at tissue for a therapeutic effect;
an infrared camera for transmitting thermal images indicative of a temperature response of the irradiated tissue based on optical properties of the irradiated tissue;
a computation circuit for applying the temperature response to a thermal laser-tissue interaction model for computing irradiation response coefficients indicative of an ablative effect on the irradiated tissue from the irradiation;
a memory for storing the thermal laser-tissue interaction model; and
a laser control responsive to the computation circuit for controlling the irradiation signal based on the irradiation response coefficients.

11. The method of claim 10 wherein the tissue properties include:
an absorption coefficient indicative of a fraction of photonic energy transferred to the tissue in the form of heat; and
and a scattering coefficient indicative of a direction change of a photon upon transferring the fraction of photonic energy.

12. The method of claim 10 wherein the ablative effect causes heating for inducing removal or cutting of the tissue.

13. The method of claim 10 wherein the irradiation signal is a laser beam and the laser control is configured to actuate the laser for aiming and traversing the laser across the tissue.

14. The method of claim 13 wherein the thermal images contain temperature information from an irradiated region of the tissue defined by the laser.

15. The method of claim 10 wherein the thermal laser-tissue interaction model is a filter for computing optical penetration of the irradiation signal into the tissue and a fraction of energy of the irradiation signal absorbed by the irradiated tissue and manifested as heat by the irradiated tissue receiving the irradiation signal.

16. The method of claim 10 wherein the filter is configured for tracking of time varying parameters based on a progression of the irradiation response coefficients in response to changes in the optical properties of the irradiated tissue resulting from the irradiation signal.

17. In an irradiation treatment environment for ablating tissue through controlled laser energy, a method for predicting a thermal response for absorption and scattering resulting from projected light, comprising:
identifying a thermal response of an irradiated tissue
receiving, from an optical sensor, thermal images indicative of a temperature response of the irradiated tissue;
applying the temperature response to a thermal laser-tissue interaction model for computing irradiation response coefficients indicative of an ablative effect on the irradiated tissue; and
controlling an irradiation source based on the irradiation response coefficients.
